(19) United States
(12) Patent Application Publication
(10) Pub. No.: US 2017/0181634 A1
(43) Pub. Date: Jun. 29, 2017

(54) MECHANICAL PALPATION FOR SUBSURFACE SENSING DEVICE

(71) Applicant: Northeastern University, Boston, MA (US)

(72) Inventors: Paige Burke, Boston, MA (US); Robert Eley, Boston, MA (US); Spencer Heyl, Boston, MA (US); Margaret McGuire, Boston, MA (US); Alan Radcliffe, Boston, MA (US)

(21) Appl. No.: 15/301,463

(22) PCT Filed: Apr. 8, 2015
(86) PCT No.: PCT/US15/24941

§ 371 Date: Oct. 3, 2016

Related U.S. Application Data
(60) Provisional application No. 61/977,303, filed on Apr. 9, 2014.

(51) Int. Cl. A61B 5/00

(52) U.S. Cl. CPC A61B 5/0053; A61B 5/4312; A61B 2562/043

(57) ABSTRACT

The present disclosure provides systems, methods and components of a mechanical palpation sensing device system. The mechanical palpation sensing device includes a plurality of probe tips. A plurality of force transfer pins extend from the plurality of probe tips. A transfer plate is coupled to the plurality of probe tips. The transfer plate houses the plurality of force transfer pins. The plurality of force transfer pins are independently slidable within the transfer plate. The mechanical palpation sensing device includes a sensor plate including a plurality of contact points. The plurality of sensors are positioned on at least one of the plurality of force transfer pins and the sensor plate intermediate the sensor plate and the plurality of force transfer pins to contact the plurality of contact points.

### CROSS REFERENCE TO RELATED APPLICATIONS

The present application claims priority to U.S. Provisional Application No. 61/977,303, filed Apr. 9, 2014, entitled “MECHANICAL PALPATION FOR SUBSURFACE SENSING DEVICE,” which application is hereby incorporated herein by reference in its entirety.

### TECHNICAL FIELD

The present application relates generally to the field of subsurface sensing devices.

### BACKGROUND

Breast cancer is currently the deadliest non-preventable form of cancer among women. Early detection of breast cancer has been shown to increase patient survival rates; therefore an early detection system that identifies tumors under the skin could help save lives.

Detection methods such as mammograms, ultrasound, and MRI scans are costly and time consuming. Additionally, tumors can pass through these exams undetected due to unique structures in an individual's breast tissue.

Tumor detection through physical palpation by a physician is another form of detection, but this method can be subjective and requires extensive training to administer and diagnose. Misread results can lead to a delay in diagnosis, putting the patient at risk.

### SUMMARY

Various embodiments disclosed herein provide systems and methods for a mechanical palpation sensing device.

In particular embodiments, the mechanical palpation sensing device includes a plurality of probe tips. A plurality of force transfer pins extend from the plurality of probe tips. A transfer plate is coupled to the plurality of probe tips. The transfer plate houses the plurality of force transfer pins. The plurality of force transfer pins are independently slidable within the transfer plate. The mechanical palpation sensing device includes a sensor plate including a plurality of contact points. The plurality of sensors are positioned on at least one of the plurality of force transfer pins and the sensor plate intermediate the sensor plate and the plurality of force transfer pins to contact the plurality of contact points.

In particular embodiments, the plurality of probe tips form a semi-cylindrical sensor tip portion. The device includes a carriage coupled to the transfer plate, in accordance with particular embodiments. The device may include an actuator coupled to the carriage. The device includes a controller coupled to the actuator, to control the location of the device in accordance with particular embodiments. The probe tips may be coupled to the transfer plate via a plurality of slidable couplings. The slidable couplings include a bolt in accordance with example embodiments. In particular embodiments, the plurality of sensors includes at least 3 sensors. The plurality of sensors may be positioned in a linear array. In particular embodiments, the plurality of sensors includes at least 5 sensors. The plurality of sensors may include a piezo-resistive force sensor configured to measure an electrical resistance. The probe tips are composed, at least in part, of a plastic in particular embodiments. The probe tips are composed, at least in part, of a polymer in particular embodiments. The polymer may include acrylonitrile butadiene styrene.

Various embodiments provide a method of manufacturing a mechanical palpation sensing device. The method includes coupling a plurality of force transfer pins to a plurality of probe tips. The method includes slidably positioning the plurality of transfer pins through a plurality of apertures in a transfer plate. The method also includes coupling the plurality of probe tips to the transfer plate. The method further includes coupling a plurality of sensors to at least one of the plurality of force transfer pins and the sensor plate intermediate the sensor plate and the plurality of force transfer pins.

In particular embodiments, the method includes coupling the carriage to the transfer plate. The method may include coupling an actuator to the carriage. The method may include coupling the plurality of sensors in at least one linear array. In particular embodiments, the plurality of probe tips are coupled to the transfer plate to move independently of one another with respect to the transfer plate.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawing primarily is for illustrative purposes and is not intended to limit the scope of the inventive subject matter described herein. The drawing is not necessarily to scale; in some instances, various aspects of the inventive subject matter disclosed herein may be shown exaggerated or enlarged in the drawings to facilitate an understanding of different features. In the drawing, like reference characters generally refer to like features (e.g., functionally similar and/or structurally similar elements).

FIGS. 1A and 1B are schematic side views of a method of detecting an object in tissue by palpation, in accordance with exemplary inventive embodiments.

FIG. 2 is a perspective view of a mechanical palpation sensing device set up to analyze a silicone phantom, in accordance with exemplary inventive embodiments.

FIGS. 3A and 3B illustrate a sensor head of a mechanical palpation sensing device, in accordance with exemplary inventive embodiments.

FIG. 4 is a schematic top view of the sensor head of FIGS. 3A and 3B.

FIGS. 5A and 5B are a side view and a top view respectively of a mechanical palpation sensing device, in accordance with exemplary inventive embodiments.

FIG. 6 is a top view of an electrical sensing circuit for use with a mechanical palpation sensing device, in accordance with exemplary inventive embodiments.

FIGS. 7A-7C are example graphs showing results of measurements of a mechanical palpation sensing device, in accordance with example embodiments.

FIG. 8 is an example graph showing results of measurements of a mechanical palpation sensing device, in accordance with example embodiments.

The features and advantages of the inventive concepts disclosed herein will become more apparent from the detailed description set forth below when taken in conjunction with the drawings.

### DETAILED DESCRIPTION

Following below are more detailed descriptions of various concepts related to, and exemplary embodiments of, inventive systems, methods and components of a mechanical palpation sensing device, in accordance with exemplary inventive embodiments.

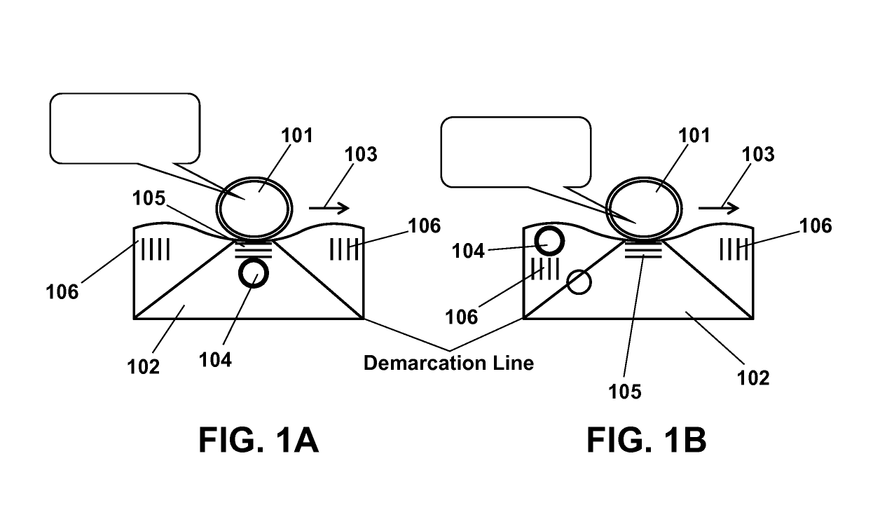

FIGS. 1A and 1B are schematic side views of a method of detecting an object in tissue by palpation, in accordance with exemplary inventive embodiments. Palpation is a physical examination to make non-invasive assessments of a patient's health. Physicians use palpation to map the underlying structures of tissues and search for abnormalities beneath the skin. Palpation combines depression of the skin and tissue with movement parallel to the skin to detect the presence of a lesion.

A mechanical palpation device 101 is depressed a depth into a tissue 102. To calibrate the device a force on the palpation device 101 when depressed the depth into the tissue 102 may be recorded as a reference value. The tissue 102 has elastic properties. After the mechanical palpation device 101 is depressed the depth into the tissue 102, the mechanical palpation device 101 is moved across the tissue in a first direction 103. As the mechanical palpation device 101 is moved across the tissue in a first direction 103, the device 101 is maintained at the depth. The depression by the mechanical palpation device 101 into the tissue 102 causes compression 105 in the tissue 102 and tension 106 in the tissue. The compression of the tissue 102 from the device 101 being depressed the depth is transferred through the tissue as the device 101 is moved. Accordingly, if the mechanical palpation device 101 moves over an object, such as a tumor or a lesion 104, the compression of the tissue will create compression 105 at the top of the lesion 104. As the mechanical palpation device 101 continues to move in the direction 103 the compression 105 at the top of the lesion 104 and the tension 106 around the lesion cause the lesion to pop or be pulled away from the compressed tissue into non-compressed tissue and generates an area of higher compression. The area of higher compression exerts a higher force on the mechanical palpation device 101, which higher force may be detected by one or more sensors as discussed further herein. The detection of the higher force helps identify the location of the lesion, which can be recognized as the location where a potentially cancerous mass is present in the tissue 102.

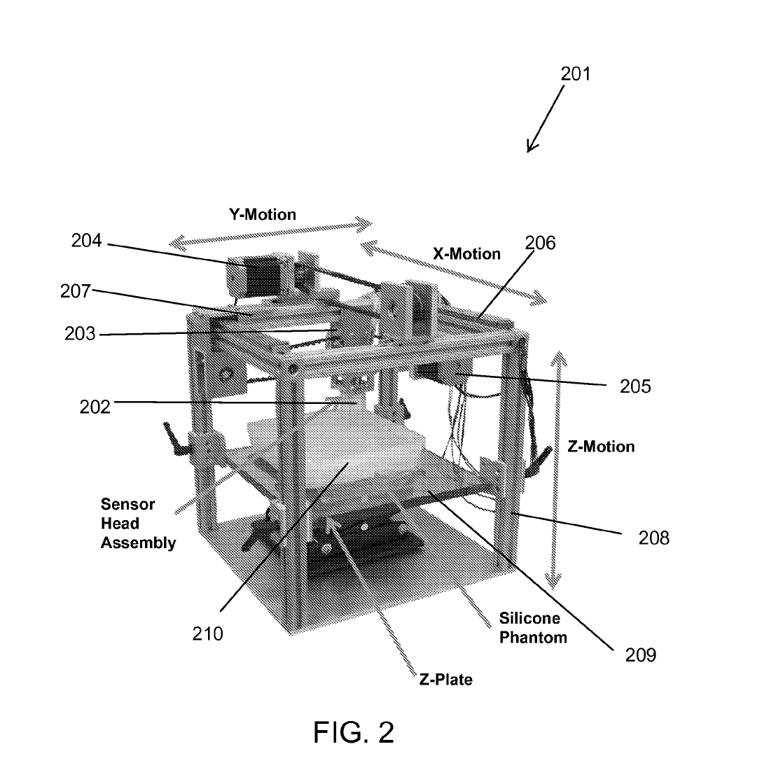

FIG. 2 is a perspective view of a mechanical palpation sensing device set up to analyze a silicone phantom, in accordance with exemplary inventive embodiments. A mechanical palpation device 201 includes a sensor head assembly 202 coupled to a carriage 203 that is configured to move in the x and y directions via actuators 204 and 205 supported on frame rails 206 and 207 of frame support 208. In the illustrated embodiment, the mechanical palpation device 201 is being used to analyze a silicone phantom 210 positioned on a z-plate 209. The silicone phantom is provided to simulate tissue, such as breast tissue of a patient. The z-plate 209 is configured to move the silicon phantom 210 along the z-axis to change the depth of depression of the sensor head assembly 202 into the silicone phantom 210. In example embodiments, the sensor head assembly 202 may be coupled to a carriage 203 configured to move in the z-axis. In example embodiments, the mechanical palpation device 201 may be positioned over an examination surface, for example positioned at the location of the z-plate, such that a patient may rest on the examination surface and the mechanical palpation device 201 may be run to examine the tissue of the patient.

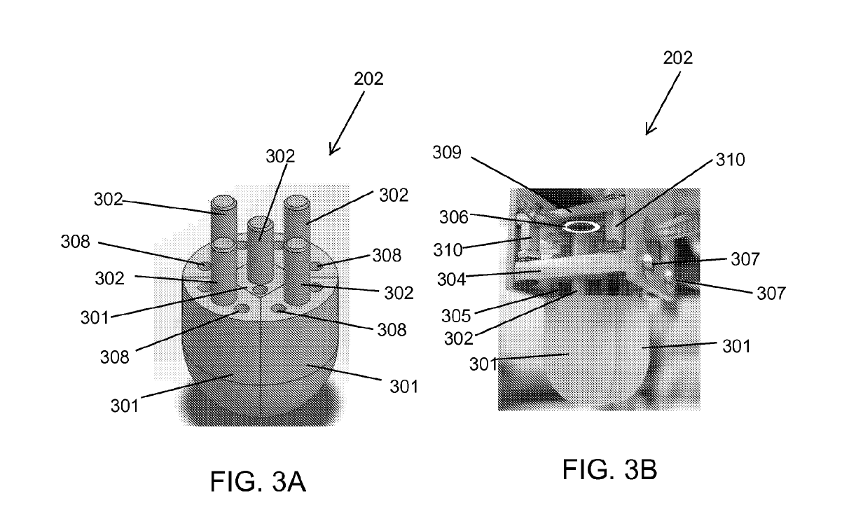

FIGS. 3A and 3B illustrate a sensor head of a mechanical palpation sensing device, in accordance with exemplary inventive embodiments. The sensor head assembly 202 is shown from above in FIG. 3A separated from the carriage 203. The sensor head assembly 202 is shown connected to the carriage 203 in FIG. 3B. The sensor head assembly 202 includes a plurality of probe tips 301 that are configured for direct contact and indentation of the tissue surface. In example embodiments, the probe tips 301 may be composed of a plastic or polymer material such as acrylonitrile butadiene styrene (ABS). The probe tips 301 may be shaped to collectively form a semi-cylindrical tip. Each of the probe tips 301 includes a force transfer pin 302 extending therefrom. The probe tips 301 include one or more apertures for housing the force transfer pins 302. In example embodiments, the force transfer pins 302 are composed of a material distinct from the probe tips 301, including, but not limited to metallic materials. The force transfer pins 302 facilitate axial sliding of probe tip 301 the sensor head assembly 202. The force transfer pins 302 also facilitate the detection of force input to the probe tips 301 by transferring the force input at the probe tip to an associated sensor. As discussed herein, in example embodiments, each of the force transfer pins 302 may include a sensor positioned on the pin 302.

As illustrated in FIG. 3A, the sensor head assembly 202 is coupled to the carriage 203, in part by the transfer plate 304, via one or more fasteners 307 The transfer plate 304 includes a plurality of holes to house the force transfer pins 302. The transfer plate 304 holds the transfer pins 302 and the probe tips 301 in place adjacent to one another and permits the transfer pins 302 to slide independently through the respective apertures in the transfer plate 304. Accordingly, as force is applied to the probe tip 301 via the tissue and or lesion, the force causes the transfer pin 302 to slide in the transfer plate 304. The probe tips 301 are slideably coupled to the transfer plate 304 via one or more fasteners 305. As shown in FIG. 3A, the probe tips 301 may include fastener openings 308 for receiving the fasteners 305. In example embodiments fastener 305 includes a bolt. The fastener opening 308 may be threaded to receive the fastener 305 in example embodiments. In example embodiments, the fastener 305 may extend upward from the probe tip 302 and the fastener 305 may include a nut removably coupled to the fastener 305 above the transfer plate 304. The nut (or alternatively head of the bolt) prevent the fastener 305 and the probe tip 302 from sliding down and out of the transfer plate 304, while still allowing the probe tip 302, the transfer pin 302, to slide upward for the force readings.

The sensor head assembly 202 also includes sensors 306. In example embodiments, the sensors include, but are not limited to piezo-resistive force sensors configured to measure an electrical resistance or a change in electrical resistance to provide a value corresponding to a change in force. As demonstrated in FIG. 3A the sensor head assembly 202 may include one or more arrays of sensors (e.g. a sensor for each force transfer pin). The sensor array may include, for example, a lead sensor, a center sensor, and a trailing sensor. The sensor array may include five sensors, such that movement in x and y plane includes a lead, center, and trailing sensor for movement along or parallel to the x-axis or the y-axis. The sensors 306 may be positioned directly on force transfer pins 302 or directly on the sensor plate 309. The sensor plate 309 is coupled to the transfer plate 304 by one or more linkages 310. The sensor plate 309 may include contact points where the force transfer pins 302 will contact the sensor plate 309 when pushed upward. The sensors 306 may be positioned at the contact points and are intermediate the sensor plate 309 and the force transfer pins 302, to measure force applied in the axial direction (e.g. along the z-axis) to determine force changes as the sensor head assembly 202 is moved along a path to determine the location of a legion in the tissue into which the probe tips 301 are pressed.

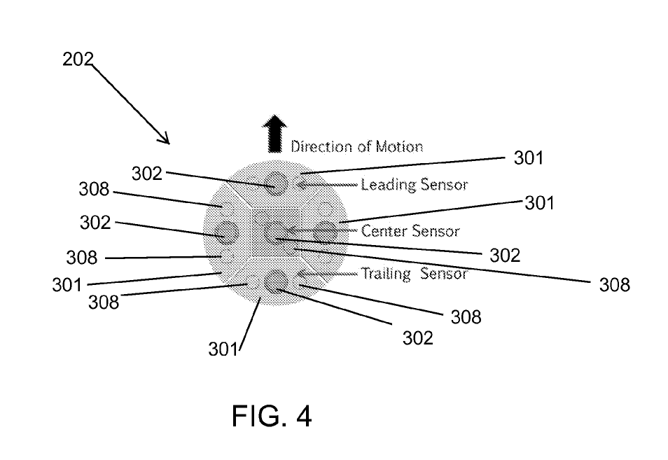

FIG. 4 is a schematic top view of the sensor head of FIGS. 3A and 3B. FIG. 4 shows an example layout of the sensor 306, the force transfer pins, 302 and the probe tips 301. While the example embodiments demonstrate a sensor head assembly with 5 sensors, example inventive embodiments may include more, less or distinct layouts other than the illustrated “t” shaped arrangement.

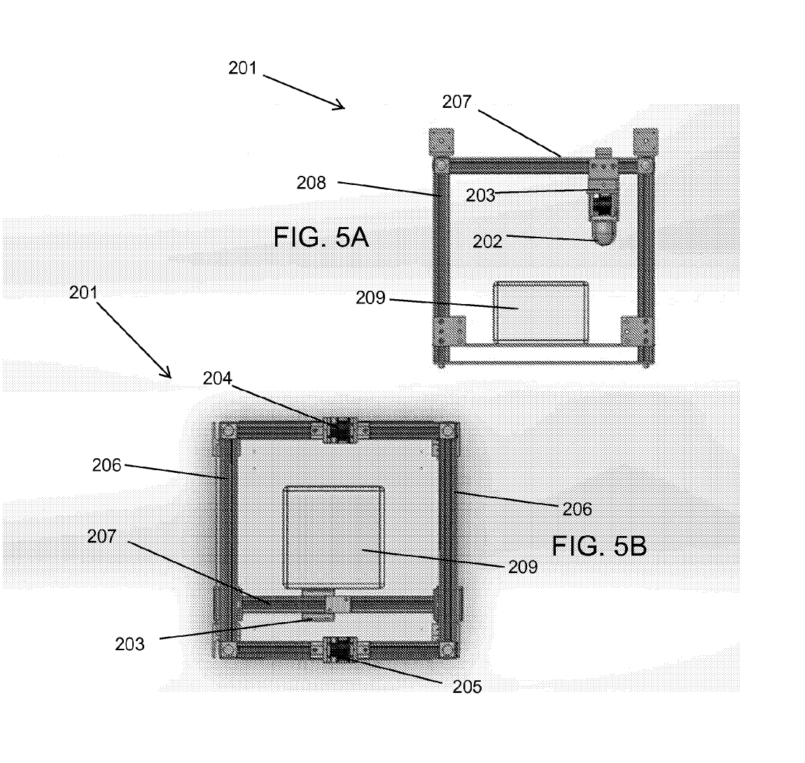

FIGS. 5A and 5B are a side view and a top view respectively of a mechanical palpation sensing device, in accordance with exemplary inventive embodiments. As shown in FIG. 5A the sensor head assembly 202 may be configured to hang down from the carriage 203 in example embodiments, to analyze a subject positioned on the z-plate 205. In example embodiments, the sensor head assembly 202, which is simply over the z-plate in FIG. 5A) may be positioned to depress tissue of a patient in a horizontal plane, for example to examine a patient while the patient is lying flat on their back.

In operation the position of the sensor head assembly 202 with respect to the tissue of a subject is moved to a certain depth within the tissue of the subject. In example embodiments, this is accomplished by moving z-plate 205 and in other examples this is accomplished by moving the sensor head 202. After the sensor head assembly 202 is moved with respect to the tissue of the subject such that the probe tips 301 are at the specified depth a first force measurement is taken as a calibration point. The sensor head assembly 202 is then moved at that depth along one or more paths through the tissue of the subject and the forces are determined and recorded. In example embodiments, the processor may be configured to flag forces that exceed a predetermined quantity or percentage threshold or change. A two dimensional plot of the readings may be generated to create a two dimensional force map of the tissue analyzed.

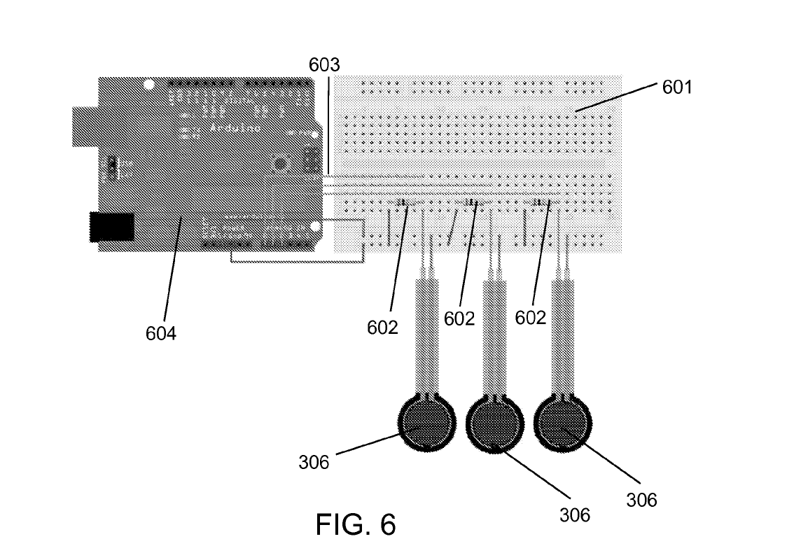

FIG. 6 is a top view of an electrical sensing circuit for use with a mechanical palpation sensing device, in accordance with exemplary inventive embodiments. As discussed herein, the sensors 306 may be coupled to one or more of the sensor plate 309 and the force transfer pins 302. The sensor 306 are configured to be intermediate the sensor plate 309 and the force transfer pin 302 to detect a force applied to the probe tips 301 and transferred through the force transfer pins 302. The sensors 306 may be electrically coupled to resisters 601, positioned on board 602. The resistors 601 are electrically coupled via board 602 and wires 603 to processor 604. Processor 604 receives inputs from the resistors to determine the force applied to a particular probe tip 301, as the sensor head assembly 202 is moved across a subject The processor may also be coupled to the one or more actuators controlling the position of the sensor head assembly 202 such that the force of each sensor may be plotted with respect to a particular position and/or time to identify the location of a lesion or other abnormality in the consistency of patient tissue.

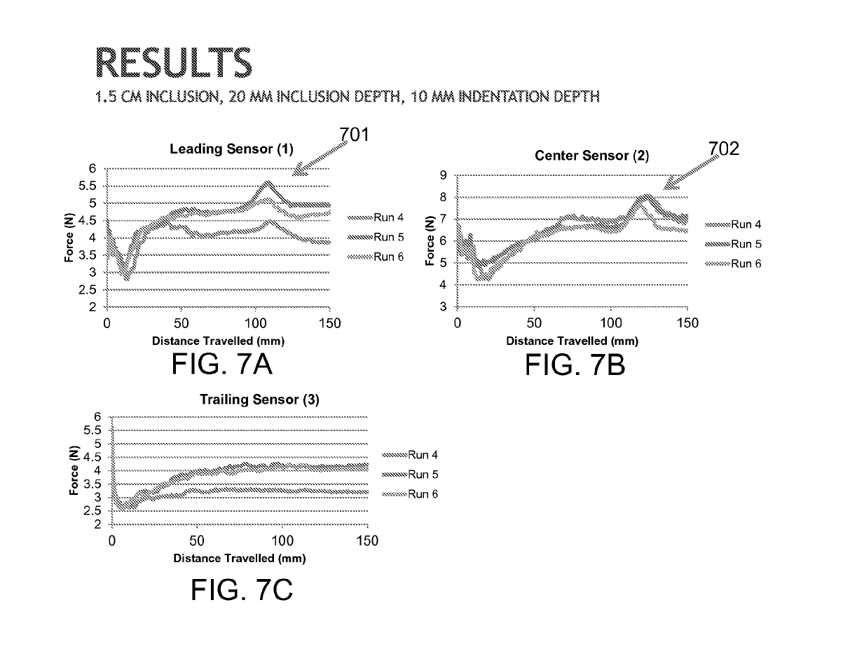

FIGS. 7A-7C are example graphs showing results of measurements of a mechanical palpation sensing device, in accordance with example embodiments. FIGS. 7A through 7C show the results of the leading, center, and trailing sensors respectively when analyzing a phantom having a lesion that is 1.5 cm in diameter, positioned 20 mm below the surface silicone phantom sample, and with the probe tips of the sensor head assembly depressed a depth of 10 mm into the surface silicone phantom sample. The graphs illustrate the distance traveled of each sensor as well as the changes in forces along the path of travel. The graph further illustrates that the lesion or inclusion was detected between 100 and 150 mm identified by increase 701 and 702.

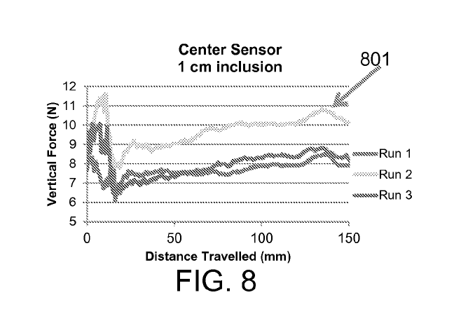

FIG. 8 is an example graph showing results of measurements of a mechanical palpation sensing device, in accordance with example embodiments. FIG. 8 shows the results of the center sensor when analyzing a phantom having a lesion that is 1 cm in diameter, positioned 20 mm below the surface silicone phantom sample, and with the probe tips of the sensor head assembly depressed a depth of 10 mm into the surface silicone phantom sample. The graph illustrates the distance traveled of the sensor as well as the changes in forces along the path of travel. The graph further illustrates that the lesion or inclusion was detected between 100 and 150 mm identified by increase 801.

As utilized herein, the terms “approximately,” “about,” “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

For the purpose of this disclosure, the term “coupled” means the joining of two members directly or indirectly to one another. Such joining may be stationary or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

Any sensor described herein may include a virtual sensor that looks up values from a non-transient memory value, receives it from a data link, from an electronic input, and/or from a hardware sensor directly measuring the value or something analogous to the value.

It should be noted that the orientation of various elements may differ according to other exemplary embodiments, and that such variations are intended to be encompassed by the present disclosure. It is recognized that features of the disclosed embodiments can be incorporated into other disclosed embodiments.

It is important to note that the constructions and arrangements of spring systems or the components thereof as shown in the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present disclosure.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, describes techniques, or the like, this application controls.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, the technology described herein may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

1. A mechanical palpation sensing device, comprising:
a plurality of probe tips;
a plurality of force transfer pins extending from the plurality of probe tips;
a transfer plate coupled to the plurality of probe tips, the transfer plate housing the plurality of force transfer pins, the plurality of force transfer pins independently slidable within the transfer plate;
a sensor plate including a plurality of contact points; and
a plurality of sensors positioned on at least one of the plurality of force transfer pins and the sensor plate intermediate the sensor plate and the plurality of force transfer pins to contact the plurality of contact points.

2. The device of claim 1, wherein the plurality of probe tips form a semi-cylindrical sensor tip portion.

3. The device of claim 1, further comprising a carriage coupled to the transfer plate.

4. The device of claim 3, further comprising an actuator coupled to the carriage.

5. The device of claim 4, further comprising a controller coupled to the actuator.

6. The device of claim 1, wherein the probe tips are coupled to the transfer plate via a plurality of slidable couplings.

7. The device of claim 6, wherein the plurality of slidable couplings includes a bolt.

8. The device of claim 1, wherein the plurality of sensors includes at least 3 sensors.

9. The device of claim 1, wherein the plurality of sensors includes a linear array of the at least 3 sensors.

10. The device of claim 1, wherein the plurality of sensors includes at least 5 sensors.

11. The device of claim 1, wherein the plurality of sensors includes a piezoresistive force sensor configured to measure an electrical resistance.

12. The device of claim 1, wherein the probe tips are composed, at least in part, of a plastic.

13. The device of claim 1, wherein the probe tips are composed, at least in part, of a polymer.

14. The device of claim 13, wherein the polymer includes acrylonitrile butadiene styrene.

15. A method of manufacturing a mechanical palpation sensing device comprising:
coupling a plurality of force transfer pins to a plurality of probe tips;
slidably positioning the plurality of transfer pins through a plurality of apertures in a transfer plate;
coupling the plurality of probe tips to the transfer plate; and
coupling a plurality of sensors to at least one of the plurality of force transfer pins and the sensor plate intermediate the sensor plate and the plurality of force transfer pins.

16. The method according to claim 15, further comprising coupling the carriage to the transfer plate.

17. The method according to claim 16, further comprising coupling an actuator to the carriage.

18. The method according to claim 15, further comprising coupling the plurality of sensors in at least one linear array.

19. The method according to claim 15, wherein the plurality of probe tips are coupled to the transfer plate to move independently of one another with respect to the transfer plate.
